(19) United States
(12) Patent Application Publication
(10) Pub. No.: US 2021/0015479 A1
(43) Pub. Date: Jan. 21, 2021

(54) SUTURE CRIMP

(71) Applicant: MWI Veterinary Supply Co., Boise, ID (US)

(72) Inventor: Graham Smith, Newburyport, MA (US)

(21) Appl. No.: 16/924,816

(22) Filed: Jul. 9, 2020

Related U.S. Application Data
(60) Provisional application No. 62/874,654, filed on Jul. 16, 2019.

(51) Int. Cl. A61B 17/04; A61F 2/08

(52) U.S. Cl. CPC A61B 17/0487; A61B 2017/00477; A61F 2002/0852; A61F 2/0811

(57) ABSTRACT

A suture crimp includes a male blade component including at least a first suture clamp and a female component receiving the male component therein and including at least a first suture bore. In a first position, the male component is received in the female component in a first engagement position such that a suture through the first suture bore in the female component is slidable relative to the male component first suture clamp. In a second position, the male component is received further in the female component such that the suture through the first suture bore in the female component is crimped by the male component first suture clamp.

### RELATED APPLICATIONS

This application claims benefit of and priority to U.S. Provisional Application Ser. No. 62/874,654 filed Jul. 16, 2019, under 35 U.S.C. §§ 119, 120, 363, 365, and 37 C.F.R. § 1.55 and § 1.78, which is incorporated herein by this reference.

### FIELD OF THE INVENTION

This subject invention relates to suture crimps.

### BACKGROUND OF THE INVENTION

In various medical and veterinary surgical procedures, a suture is extended through a crimp, through a bone, for example, and ultimately back through the crimp. The suture must be tightened and then the crimp deformed. Mechanically deformable crimps must be crushed by a mechanical pliers assembly often requiring a great deal of force sometimes on the order of 5KN (1100 lbf). Some pliers employed to compress the crimps required significant mechanical advantage and can be unwieldy and complex.

In one example, in a canine cranial cruciate ligament stabilization procedure, a suture is threaded through a hole drilled in the tibial tuberosity, looped around the lateral fabella, and then the two fre ends of the suture are tensioned and crimped. See U.S. Pat. No. 6,395,010 incorporated herein by this reference.

This procedure takes three crimps, two separate tools (a tensioning device and a crimper) and at least two people to complete the procedure.

### BRIEF SUMMARY OF THE INVENTION

Featured is a new crimp which requires significantly less force to suitably crimp suture material. Also featured is a new cranial cruciate suture.

In one embodiment, a suture crimp female component includes a channel therein and one or more suture bores extending into the channel. A male blade component is slidable into the female component channel and includes one or more suture clamps each cooperating with a suture bore to crimp suture material extending through the suture bores. A key and lock arrangement is configured to fix the male blade component relative to the tubular female component in a first position allowing the suture material to slide relative to the crimp to tension the suture and also to fix the male blade component relative to the tubular female component in a second position as the male blade component is pressed further into the female component channel to properly crimp the suture material.

Also featured is a suture crimp comprising a male component including at least a first suture clamp and a female component receiving the male component therein and including at least a first suture bore. A key is associated with one of the male and female components and a lock is associated with the other of the male and female components. The key and the lock are preferably configured in a first position to engage the male component in the female component in a first engagement position wherein a suture through the first suture bore in the female component is slidable relative to the male component first suture clamp. The key and lock are configured in a second position to engage the male component in the female component in a second engagement position wherein the suture through the first suture bore in the female component is crimped by the male component first suture clamp.

The male component, in one design, includes first and second suture clamps and the female component includes corresponding first and second suture bores. The male component may include two keys and the female component may include two corresponding locks. In one version, each key includes an ear with first and second teeth and each lock includes a ledge engaging the first tooth in the first position and engaging the second tooth in the second position. Each ear is preferably flexible and biased to engage the lock. The first suture clamp may include a concave opening extending through the male component. In one version, the female component includes a channel receiving the male component therein between the front and rear walls and the first suture bore includes concave openings in the front and rear walls extending into the channel. Preferably the first suture clamp concave opening is oriented the opposite of the concave openings in the front and rear walls of the female component. The female component may include right and left sidewalls each with a lock. The male component may include a first key with an outwardly biased ear on the right side of the male component having first and second teeth engagable with the right female component sidewall lock and a second key comprising an outwardly biased ear on the left side of the male component having first and second teeth engagable with the left female component sidewall lock.

In another version, the female component includes top and bottom passages and the lock includes at least two ledges extending into each passage and the male component key includes top and bottom ears slidable in said respective top and bottom passages and each with at least a first and second tooth engaging a respective first and second ledge. Also, the first suture clamp and/or the first suture bore may include suture material gripping features.

Also features is a suture crimp comprising a female component including a channel therein and front and rear suture bores opening into the channel and a male blade component slidable into said female component channel and including at least one suture clamp cooperating with said suture bores to crimp suture material extending through said suture bores.

Also featured is a ligament stabilization suture comprising a suture passed through a suture crimp as described above and located on the medial side of the tibia, then passed through a first hole in the tibia, then passed over the fabella of the femur, then passed through the first hole in the tibia or a second hole in the tibia, and then passed through the suture crimp and tightened while the suture crimp is closed about the suture.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIGS. 1A-1C are schematic views showing a prior art ligament stabilization procedure where three crimps and two tools are required in order to complete the procedure;

FIGS. 2A-2B are highly schematic views showing the theory of operation of various new crimps associated with the subject invention;

FIGS. 3A-3B are highly schematic views showing alternative arrangements of the crimp bore and the clamp member,

FIG. 4A is a schematic view showing one particular version of a crimp where the male blade portion is positioned relative to the female component allowing the suture ends to slide relative to the crimp for suture tensioning;

FIGS. 4B-4C are schematic cross-sectional views showing the version of the crimp shown in FIG. 4A where the male blade portion is positioned relative the female component allowing the suture ends to slide relative to the crimp for suture tensioning;

FIG. 4D is a schematic view showing how the male blade member has now been pushed further into the female component to crimp the two suture ends;

FIG. 5A is a schematic view showing another version of a crimp with only a single male suture clamp showing the position where the male component is positioned relative to the female component allowing suture material to slide relative to the crimp;

FIG. 5B is a schematic view showing the crimp of FIG. 5A where the male member has now been pressed further down into the female member to crimp the suture material;

FIG. 6A is a schematic view showing another version of a crimp where the male component slides axially into the female component with FIG. 6A showing the male component locked in place relative to the female component allowing the suture material to be tensioned;

FIG. 6B is a schematic view showing the crimp of FIG. 6A where the male component has now been slid further into the interior channel of the female component and locked in place with respect thereto properly crimping the suture material;

FIGS. 7A-7C are schematic views showing the version of the crimp shown in FIGS. 4A-4D used in a ligament stabilization suture technique;

FIGS. 8A-8B are schematic views of another embodiment of a suture crimp;

FIGS. 9A-9C are schematic views of another embodiment of a suture crimp; and

FIG. 10 is a schematic view of a tool useful for closing the new crimp.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

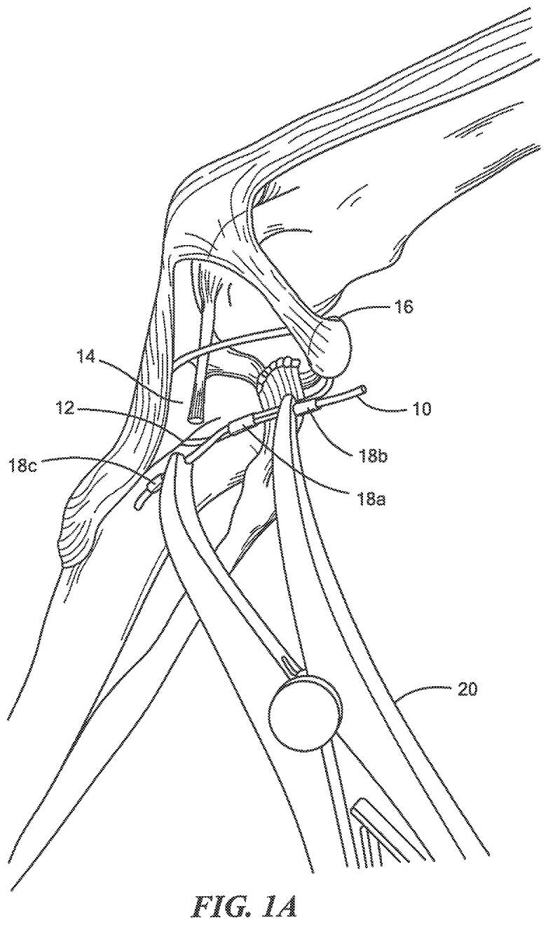

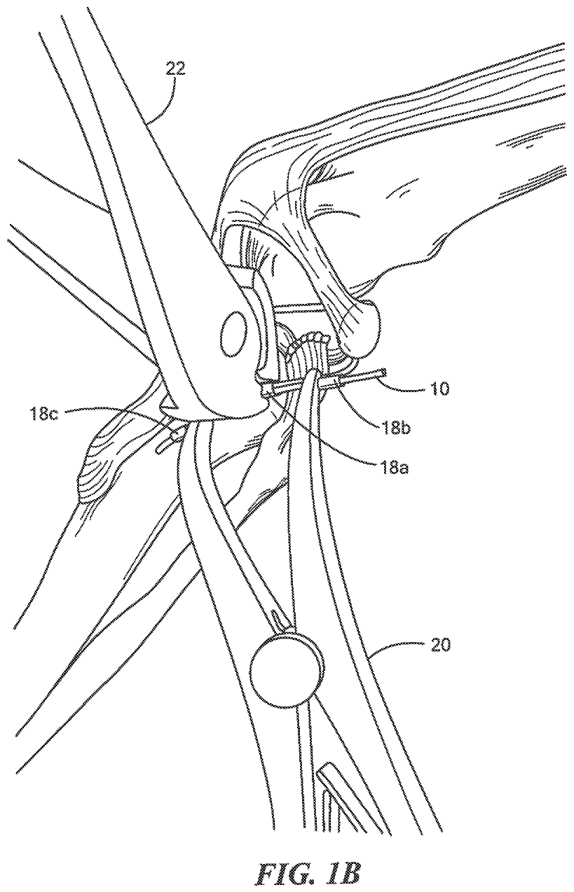

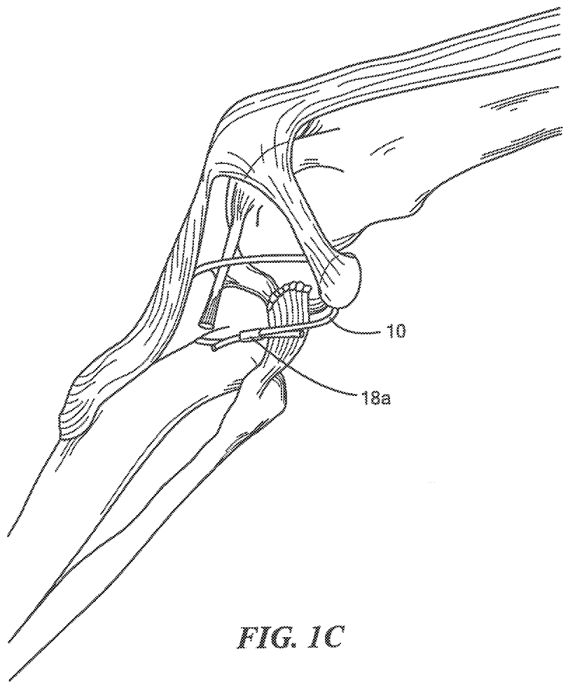

As shown in FIGS. 1A-IC, in one example, a prior canine cranial cruciate ligament stabilization procedure entails a suture 10 threaded through a hole 12 drilled in the tibial tuberosity 14, then looped around the lateral fabella 16, and then both ends of the suture are threaded through crimp 18a. Crimps 18b and 18c are secured to the two ends of suture 10 and tensioning device 20 is used to tighten the suture. In FIG. 1B, crimping device 22 is used to deform crimp 18a as shown in FIG. 1C.

This procedure may require one person to operate the tensioning device 20 and another person to operate crimper 22. And, if crimp 18a is not sufficiently deformed, the suture can loosen. Alternatively, if the crimp is over deformed, the suture can break. In general, it can very difficult for some veterinarians and other medical professionals to properly operate crimper 22 to sufficiently deform the crimps.

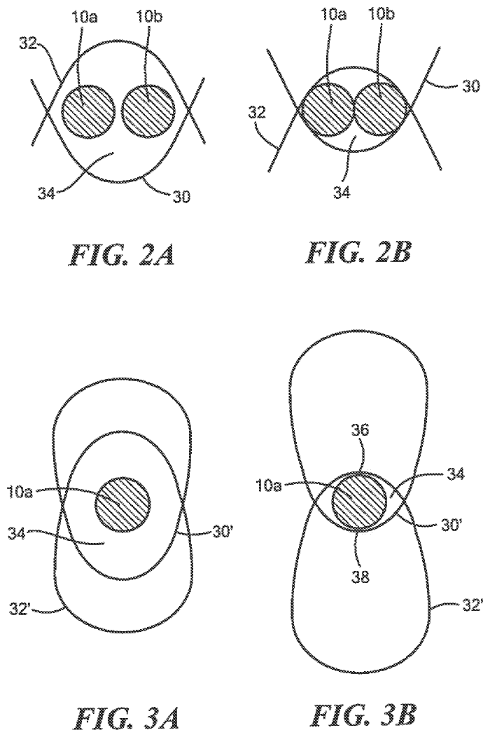

In one aspect, the invention features, in one preferred embodiment, a suture bore member 30, FIGS. 2A-2B and a suture clamp member 32 which in the position shown in FIG. 2A defines an opening 34 larger than the combined diameter of suture ends 10a and 10b (or only one suture end in some embodiments) enabling the suture ends 10a and 10b to be tensioned as in the position shown in FIG. 2A wherein the two suture ends 10a, 10b can slide with respect to opening 34. In the position shown in FIG. 2B, suture clamp member 32, typically associated with a crimp male member pressed into a crimp female member including suture bore member 30, is forced downward and fixed into a position such that now opening 34 is smaller than the combined diameters of suture ends 10a, 10b thus crimping the suture ends. Similarly, FIG. 3A shows overlapping tear drop shaped suture bore member 30′ and suture clamp member 32′ oppositely orientated so that in the cooperative position shown in FIG. 3A, suture end 10a is free to slide in opening 34 and, in the cooperative position shown in FIG. 3B, suture end 10a is crimped between concave portion 36 of suture clamp member 32′ and concave portion 38 of suture bore member 30′.

In this way, suture material can be tensioned manually by pulling on the ends of two sutures or a single suture with one hand and then, by driving the crimp clamp member relative to the suture bore member (using a small needle nose pliers, for example), the suture or sutures are properly crimped. Much less force is needed compared to deforming a standard metal ferrule type crimp. And, the novel crimp ensures the suture material is crimped to the extent that the crimp locks the suture material in place without over crimping the suture material.

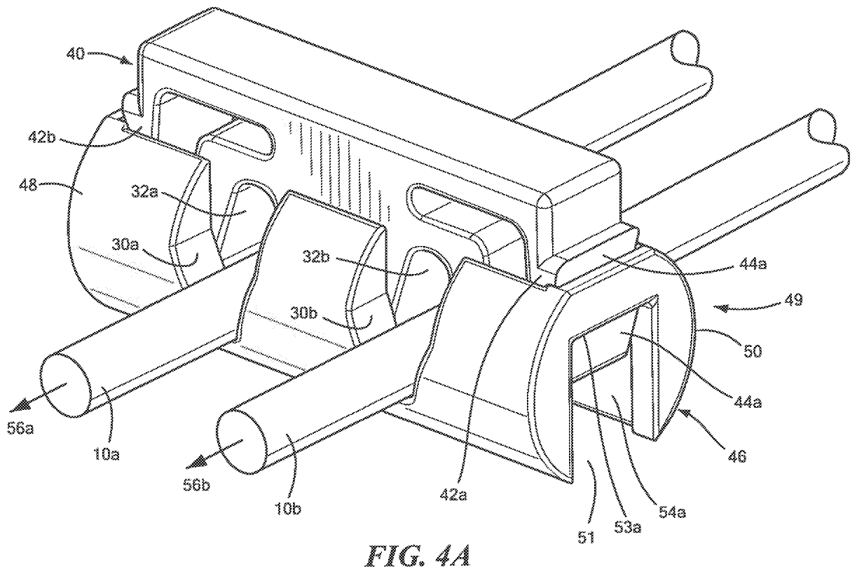

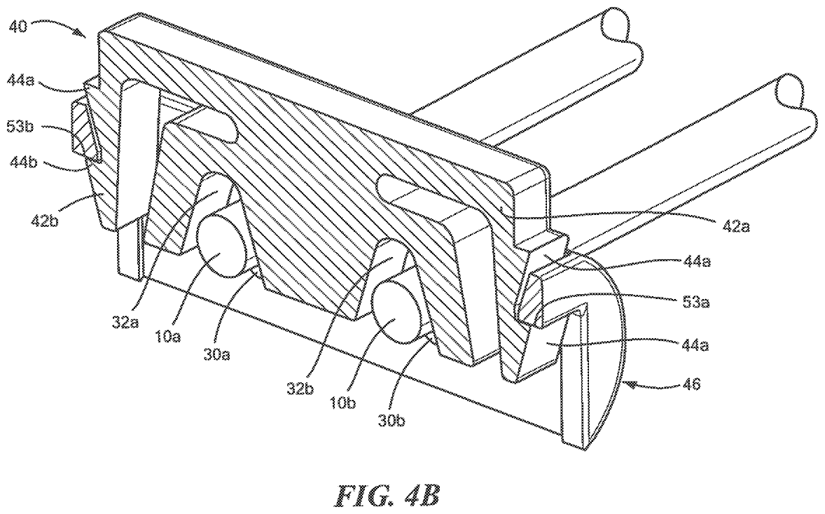

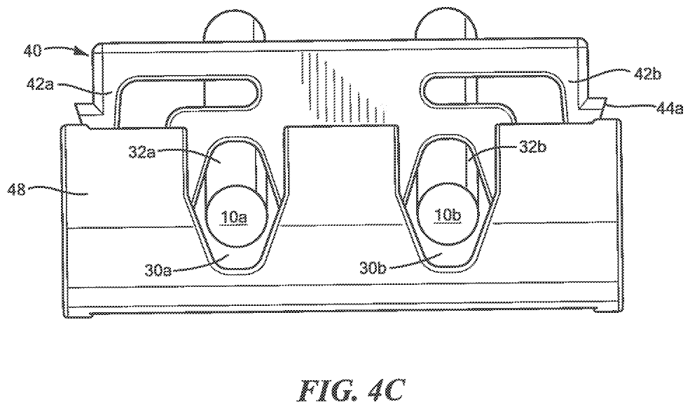

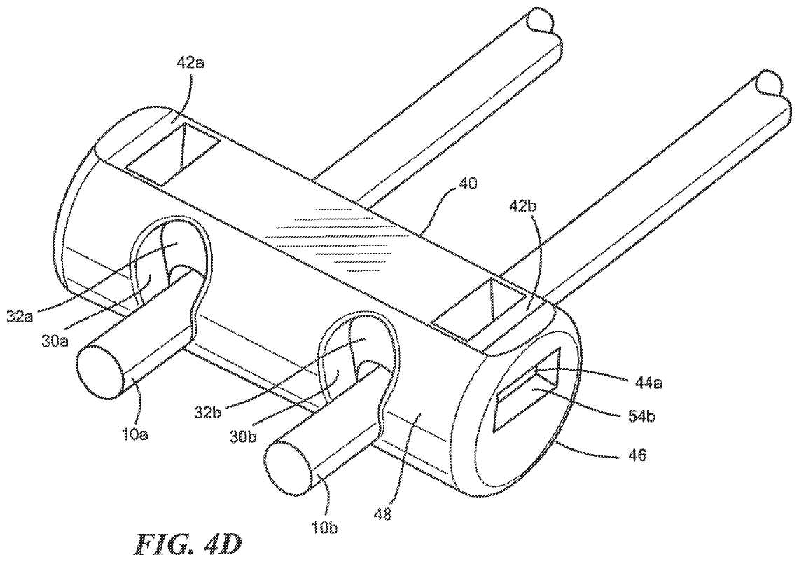

FIGS. 4A-4D show a version where crimp 49 male blade member 40 includes two side by side suture clamps 32a, 32b each in the approximate shape of an upside down U opening through the male blade member 40 body from front to back. The suture clamp openings are preferably wider at the bottom and narrower at the top. On each side of male blade member 40 are outwardly biased flexible key ears 42a, 42b each with one or more teeth 44. Male blade member 40 is received in a channel 51 in the tube shaped crimp female component 46. This channel may extend through from the top and optionally through the bottom of the female component. Female component 46 includes side by side suture bores 30a and 30b extending through front wall 48 into the central channel and a corresponding set of suture bores extending through the back wall 50 and into the central channel. These suture bores may be approximately the shape of a U defining openings wider at the top and narrower at the bottom.

In this way, suture ends 10a and 10b each extend in the same direction through the female component 46 back wall bores, through the male component 40 suture clamp openings 32a, 32b, and through the female component front wall bores 30a, 30b. When male component 40 is in the position shown in FIGS. 4A-4C, with optional lower teeth 44b of the flexible outwardly biased ears 42a, 42b locked in he respective end wall lock channels 54a, 54b of female component 46, suture ends 10a and 10b are free to slide relative to the crimp and can be tightened by hand (or by using a tool) by pulling on the sutures in the direction shown by arrows 56a, 56b. When the approximate tension has been applied to the sutures, FIG. 4D shows how the male blade member 40 is pushed further down into the female component 46 locking top teeth 44a of outwardly biased flexible ears 42a, 42b in the side locks of the female component 46 whereupon clamps 32a, 32b apply the appropriate force to suture ends 10a, 10b in cooperation with the female component 46 bores 30a, 30b. Top tooth 44a in this example engages top ledge 53b of lock opening 54b in the female component.

Thus, in FIGS. 4A-4C the suture ends pass through the wider portions of the male suture clamp openings and the wider portions of the female component 46 suture bores to allow the sutures to be tensioned and in FIG. 4D the suture material passes through the narrower portions of the male suture clamp openings and the narrower portions of the female component 46 suture bores to crimp the suture ends.

In one example, the crimp is fairly small, for example 0.300 inches long and 0.120 inches in diameter. The crimp may be molded from metal or plastic. In other examples, the male and female components may take other forms as may the suture clamps and bores.

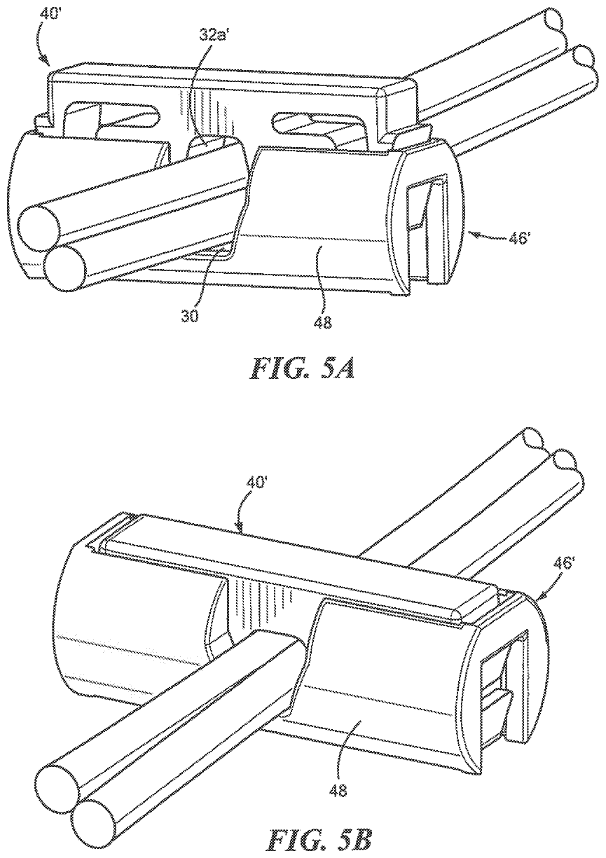

FIGS. 5A-5B show an alternative crimp with female component 46′ including only one set of front wall and rear wall suture bores (see front wall bore 30) and male component 40′ includes only a single suture clamp 32a′ for one suture or two suture ends as shown.

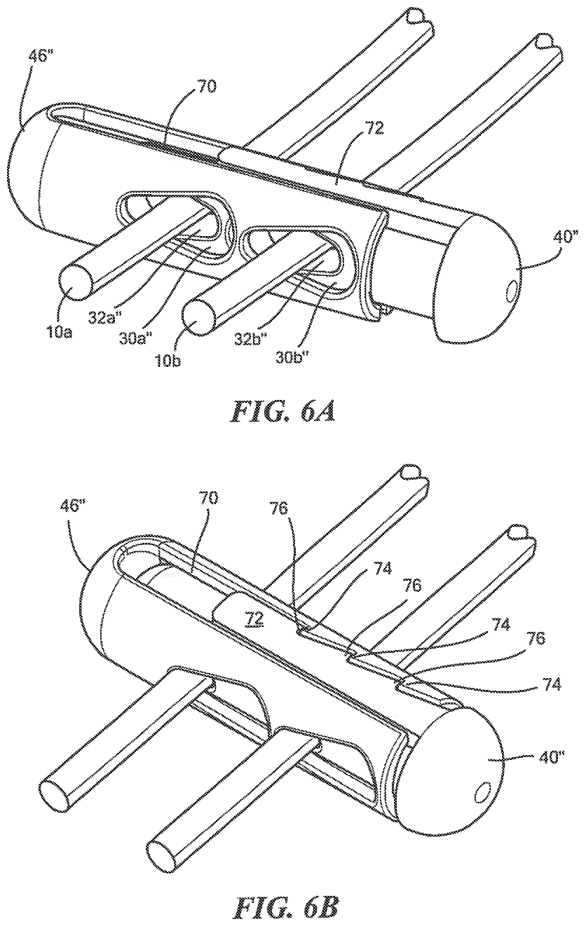

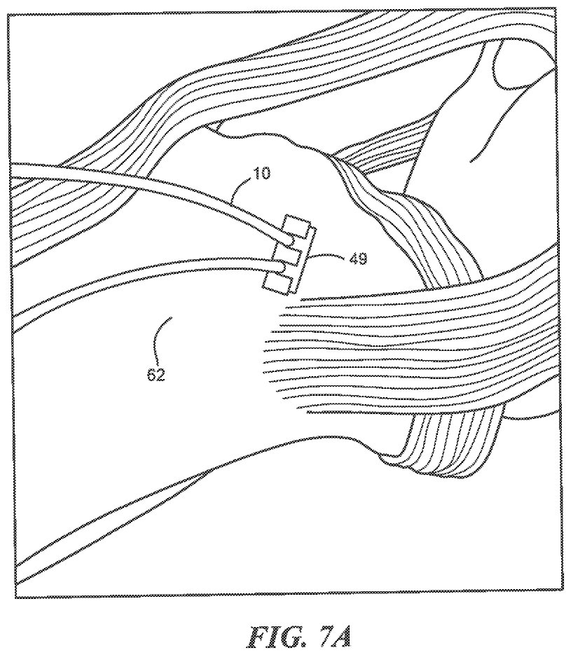

FIGS. 6A-6B show another example where male member 40″ slides axially into one end of female component 46″ via a longitudinal channel within the female component. Here the lock and key mechanisms may include a top and bottom longitudinal passages in female component 46′(see top passage 70) serving as the lock and male component 40″ includes top and bottom longitudinally extending ears (see top ear 72). Top passage 70 ledges 74 cooperate with top ear teeth 76 to lock the male component 40″ relative to the female component 46″, as shown in FIG. 6A, to allow tensioning of the suture material and to lock male component 40″ relative to female component 46″ as male component 40″ is slid further into female component 46″ as shown in FIG. 6B to properly crimp the suture material. The same is true for the bottom female component 46″ passage and male component 40″ longitudinal ear. In this version, there may be one or more sets of bores and one or more corresponding suture clamps.

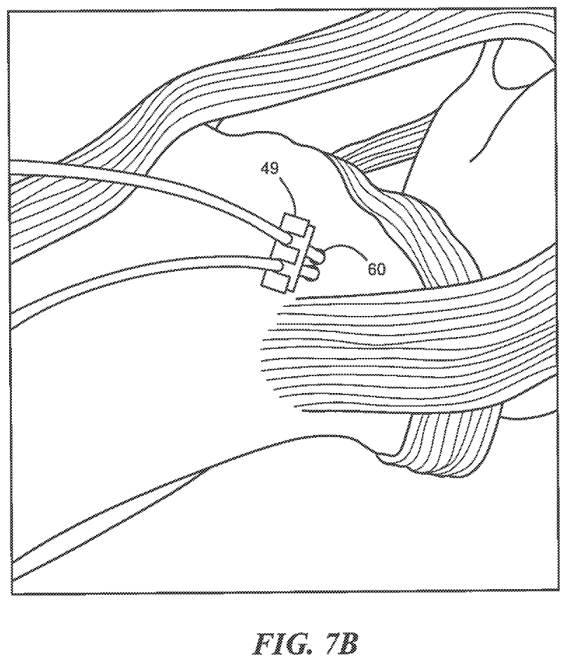

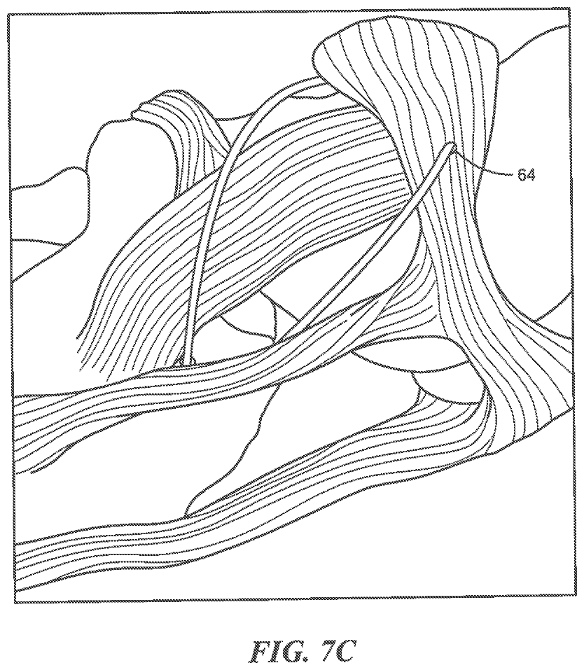

FIGS. 7A-7C show an example of a ligament suture featuring crimp 49. Suture 10 passes, for example, through crimp 49 placed on the medial side of tibia 62, so as not to interfere with any ligament, and then back through crimp 49 through one or more holes 60 in the tibial tuberosity, through ligament portions 64, over the fabella of the femur, and back through the same or another hole 60 in the tibial tuberosity.

The benefits include an easier to use crimp, a crimp which requires significantly less force to deploy, crimps deployed perpendicular to the axis of the suture and thus better suited to sit flush on the tibial tubercle, and crimps which can be deployed with less force by simple pliers or a custom deployment instrument.

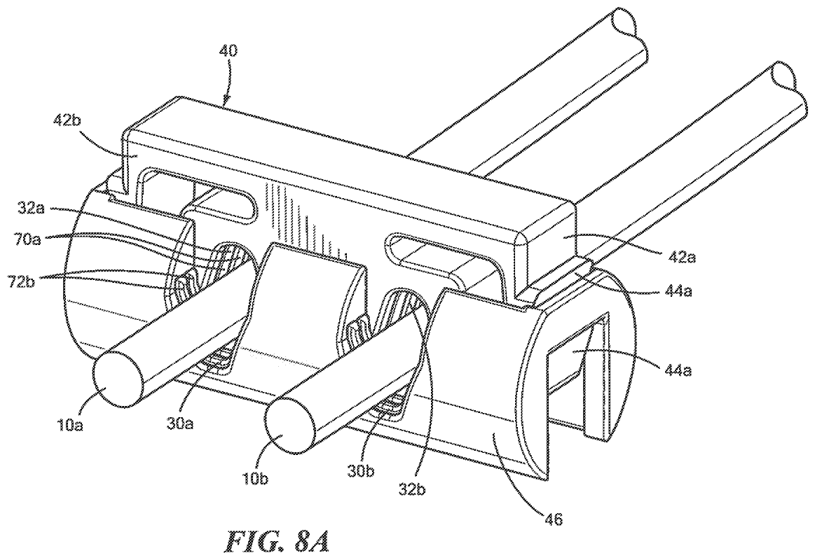

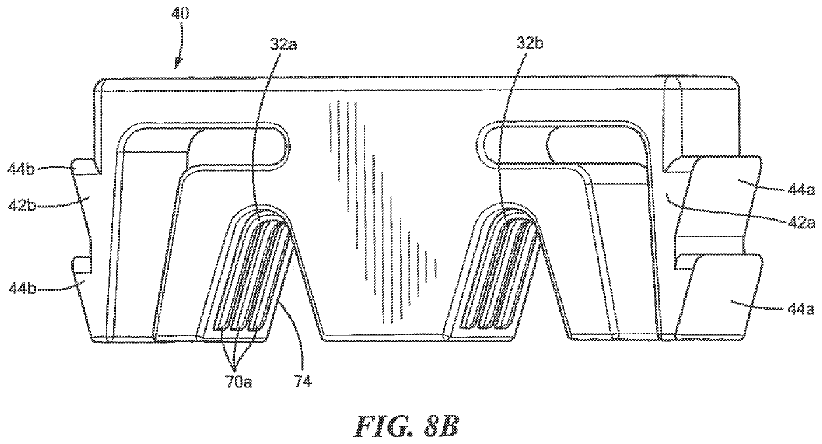

FIGS. 8A-8B show another embodiment where suture clamps 32a, 32b include suture gripping features 70a (e.g., spaced ribs on the inside wall 74 of each clamp). Similar suture gripping features 72b may be associated with suture bores 30a, 30b.

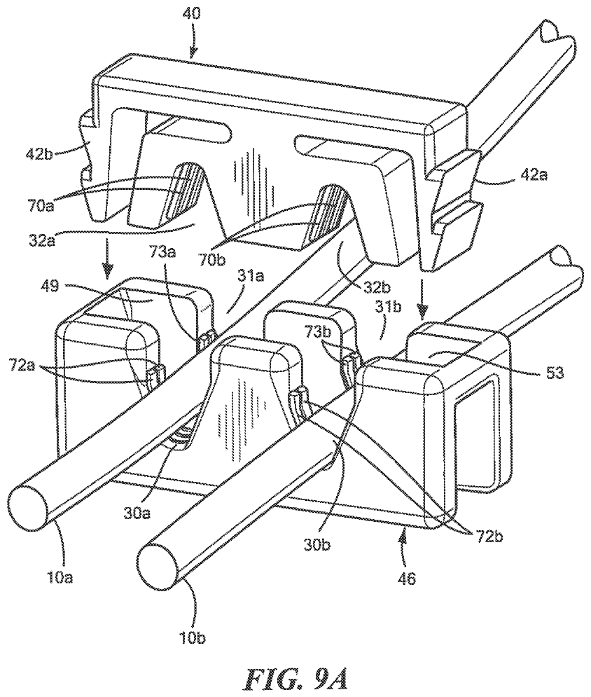

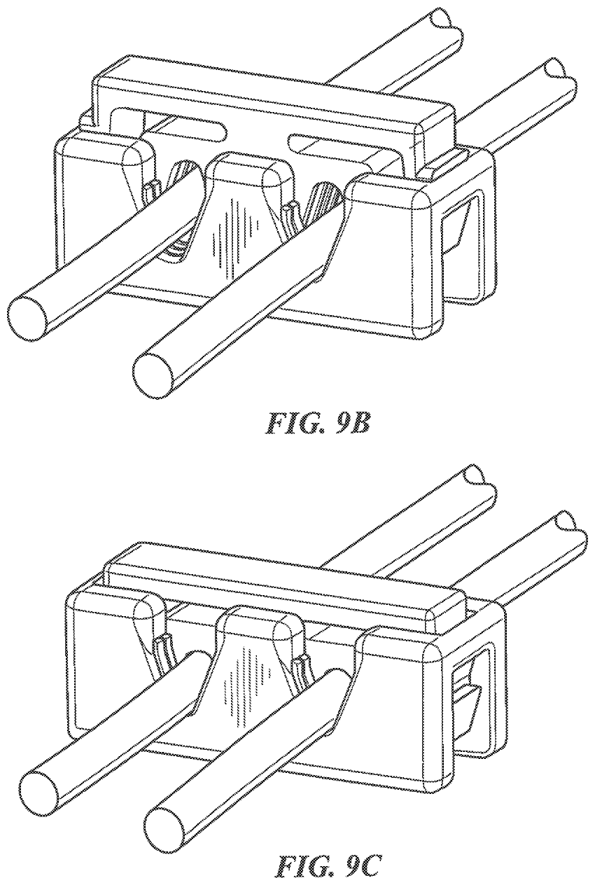

FIGS. 9A-9C show another example of crimp male blade member 40 and the female component 46. Male blade member 40 includes suture clamps 32a. 32b with suture gripping features 70a, 70b, respectively, and key ears 42a, 42b. Male blade member 40 is received in channel 53 of female component 46 which includes suture bores 30a, and 30b in front wall 48 each also including suture gripping features 72a, 72b, respectively, and suture bores 31a, 31b in back wall 49 both also including suture gripping features 73a, 73b, respectively.

FIG. 9A shows the male blade member before insertion into the female suture component, FIG. 9B shows the male component locked into the female suture component in first position where the sutures are freely slidable with respect to the crimp, and FIG. 9C shows the male component inserted deeper into and locked with respect to the female suture component in a second position where the sutures are crimped by the crimp.

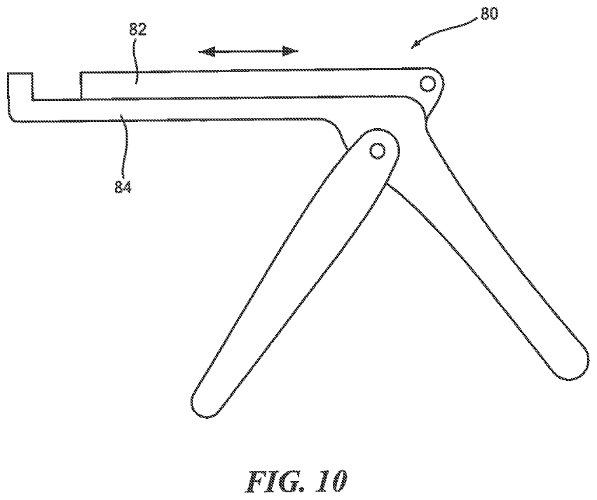

FIG. 10 shows a tool 80 which may be used to engage the male blade and suture female components to crimp the suture as sliding jaw 82 moves relative to fixed jaw 84 to drive the male blade member deeper into the female suture component.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A suture crimp comprising:
a male component including at least a first suture clamp;
a female component receiving the male component therein and including at least a first suture bore;
a key on one of the male and female components;
a lock on the other of the male and female components receiving the key;
the key and the lock configured in a first position to engage the male component in the female component in a first engagement position wherein a suture through the first suture bore in the female component is slidable relative to the male component first suture clamp; and
the key and lock configured in a second position to engage the male component in the female component in a second engagement position wherein the suture through the first suture bore in the female component is crimped by the male component first suture clamp.

2. The crimp of claim 1 in which the male component includes first and second suture clamps.

3. The crimp of claim 2 in which the female component includes corresponding first and second suture bores.

4. The crimp of claim 1 in which the male component includes two keys and the female component includes two corresponding locks.

5. The crimp of claim 4 in which each key includes an ear with first and second teeth and each lock includes a ledge engaging the first tooth in the first position and engaging the second tooth in the second position.

6. The crimp of claim 5 in which each ear is flexible and biased to engage the lock.

7. The crimp of claim 1 in which the first suture clamp includes a concave opening extending through the male component.

8. The crimp of claim 7 in which the female component includes a channel receiving the male component therein between the front and rear walls and the first suture bore includes concave openings in the front and rear walls extending into the channel.

9. The crimp of claim 8 in which the first suture clamp concave opening is oriented the opposite of the concave openings in the front and rear walls of the female component.

10. The crimp of claim 8 in which the female component includes right and left sidewalls each with a lock.

11. The crimp of claim 10 in which the male component includes a first key with an outwardly biased ear on the right side of the male component having first and second teeth engagable with the right female component sidewall lock and a second key comprising an outwardly biased ear on the left side of the male component having first and second teeth engagable with the left female component sidewall lock.

12. The crimp of claim 9 in which the female component includes top and bottom passages and the lock includes at least two ledges extending into each passage and the male component key includes top and bottom ears slidable in said respective top and bottom passages and each with at least a first and second tooth engaging a respective first and second ledge.

13. The crimp of claim 1 in which the first suture clamp and/or the first suture bore includes suture material gripping features.

14. A suture crimp comprising:
a female component including a channel therein and front and rear suture bores opening into the channel; and
a male blade component slidable into said female component channel and including at least one suture clamp cooperating with said suture bores to crimp suture material extending through said suture bores.

15. The suture crimp of claim 14 further including a key and lock arrangement associated with the female component and male blade component and configured to fix the male blade component relative to the female component in a first position allowing the suture material to slide relative to the crimp and to fix the male blade component relative to the female component in a second position crimping the suture material.

16. The suture crimp of claim 14 in which the male blade component includes first and second side by side suture clamps.

17. The suture crimp of claim 16 in which the female component includes corresponding first and second sets of front and rear suture bores.

18. The suture crimp of claim 15 in which the male component includes at least two keys and the female component includes at least two corresponding locks.

19. The suture crimp of claim 18 in which each key includes an ear with first and second teeth and each lock includes a ledge engaging the first tooth in the first position and engaging the second tooth in the second position.

20. The suture crimp of claim 19 in which each ear is flexible and biased to engage a lock.

21. The suture crimp of claim 14 in which the first suture clamp includes a concave opening extending through the male component.

22. The crimp of claim 15 in which the female component includes right and left sidewalls each with a lock.

23. The suture crimp of claim 15 in which the male blade component includes a first key with an outwardly biased ear on the right side of the male component having first and second teeth engagable with the right female component sidewall lock and a second key comprising an outwardly biased ear on the left side of the male component having first and second teeth engagable with the left female component sidewall lock.

24. The suture crimp of claim 14 in which the front and/or rear suture bores and/or the suture clamp include a suture material gripping feature.

25. A ligament stabilization suture comprising:
a suture passed through a suture crimp located on the medial side of the tibia;
then passed through a first hole in the tibia;
then passed over the fabella of the femur;
then passed through the first hole in the tibia or a second hole in the tibia; and
then passed through the suture crimp and tightened while the suture crimp is closed about the suture.

26. The suture of claim 25 in which the suture crimp includes:
a female component including a channel therein and front and rear suture bores extending into the channel; and
a male blade component slidable into said female component channel and including at least one suture clamp cooperating with said suture bores to crimp the suture material extending through said suture bores.

27. The suture of claim 25 further including a key and lock arrangement associated with the female component and male blade component and configured to fix the male blade component relative to the female component in a first position allowing the suture material to slide relative to the crimp and to fix the male blade component relative to the female component in a second position crimping the suture material.

28. The suture of claim 25 in which the front and/or rear suture bores and/or the suture clamp include a suture material gripping feature.

29. A suture crimp comprising:
a male blade component including at least a first suture clamp;
a female component receiving the male component therein and including at least a first suture bore;
the male blade component and the female component configured to engage the male blade component in the female component in a first engagement position such that a suture through the first suture bore in the female component is slidable relative to the male blade component first suture clamp; and
the male blade component and the female component configured in a second position to engage the male component deeper in the female component in a second engagement position such that the suture through the first suture bore in the female component is crimped by the male blade component first suture clamp.
